(19) United States
(12) Patent Application Publication
(10) Pub. No.: US 2008/0065029 A1
(43) Pub. Date: Mar. 13, 2008

(54) NERVE BLOCK NEEDLE AND RELATED METHODS

(76) Inventor: N. Sandor Racz, Coppell, TX (US)

Correspondence Address: WINSTEAD PC, P.O. BOX 50784, DALLAS, TX, 75201, US

(21) Appl. No.: 11/853,785

(22) Filed: Sep. 11, 2007

Related U.S. Application Data
(60) Provisional application No. 60/843,723, filed on Sep. 11, 2006.

(51) Int. Cl. A61M 5/32; A61M 31/00; A61M 5/00

(52) U.S. Cl. 604/272; 604/264; 604/95.02

(57) ABSTRACT

A novel sharp needle with an at least partially occluded, closed distal and at least one side port and related methods is disclosed. Various embodiments of needles of the present invention provide for at least one benefit of enhanced injection characteristics, increased operational efficiency, reduced cost per unit, reduced incidence of injury through intraneural/intravascular injection, reduced incidence of injury through pricking/piercing, and/or the like.

### RELATED APPLICATIONS

This application claims priority to, and incorporates by reference U.S. Provisional Patent Application No. 60/843,723 filed Sep. 11, 2006.

### SUMMARY OF THE INVENTION

Needles with orifices located along the length of the needles are known in the art. P. C. Lund, M.D. in his book Principles and Practices of Spinal Anesthesia, Charles C. Thomas Books (1971) discloses a variety of needles used for a spinal anesthesia The Hingson-Ferguson Spinal Needle (pgs. 280 and 281) and the Lemmon Needle (pgs. 287 and 288) are needles having openings both at the needle distal and along the length of the needle side. This configuration is stated as reducing the chance of the inability of administering the anesthesia due to the clogging of the opening at the needle distal end. However, an opening at the distal end can result in an improper injection, such as, but not limited to an intraneural injection and/or an intravascular injection. Accordingly, certain benefits would be experienced from a needle without an opening or orifice in the tip that still is capable of delivering a medicament to a desired tissue.

A typical needle includes four integral elements: a proximal “hub” or connector for fluidic attachment to a medical instrument; a straight tubular “shaft” which is typically at least partially hollow; a conical or tapered portion at the distal end of the straight tubular shaft portion; and a distal end or “tip”. In addition, many hypodermic needles further have a removable cap that fits around the tip and shaft to removably frictionally engage the hub. However, none of these features, including the cap, increases user safety during needle use or operation. Because it typically entails placing one's hand in front of the needle tip, the capping procedure may be so risky that many experts recommend that it not be performed (and that exposed needles be transported to containers for sharp disposal). Accordingly, certain benefits would be found in a needle with an at least partially occluded tip that was still able to engage and/or enter at least one tissue of a patient without the assist of an introducer.

A very successful embodiment of a needle with an opening at the distal portion and with a side port is disclosed in U.S. Pat. No. 5,817,074 (“the '074 patent”). The '074 patent discloses a stellate ganglion sympathetic block needle having a side port positioned at a predetermined distance from the needle distal. The stellate ganglion sympathetic block needle allows for an effective stellate ganglion sympathetic block even if the needle is placed such that the needle distal opening is under the anterior longitudinal ligament, which results in the needle distal opening being, constricted, thereby interfering with the injection of the anesthesia. When this interference occurs, the side port of the novel stellate ganglion sympathetic block needle allows directional injection onto the surface of the anterior longitudinal ligament in order to spread onto the surface of the longus coli muscle toward the stellate ganglion thereby achieving an effective stellate ganglion sympathetic block. This invention has found wide applicability, however, certain procedures might prefer or require a needle without an opening at the distal portion.

Further, it is known that needles with a blocked tip (distal end) and with a side opening are used for procedures such as thoracentesis which involves inserting the needle through the thoracic cage into the pleural space between the lung and the chest wall to draw off fluid for diagnostic or therapeutic purposes. Thoracentesis needles generally consist of an orifice free, sharp conical end and a circular side hole for draining fluid.

U.S. Pat. No. 5,573,519 discloses an elongated, hollow spinal needle having a modified pencil-like point with a rounded shoulder at the juncture between modified, pencil-like point and the body of the spinal needle. A side port formed in the hollow needle is adjacent the rounded shoulder, extending from the rounded shoulder. Further the leading edge of the side port is located not more than 1.5 times the external diameter of the hollow needle from the tip (distal end) of the pencil-like point to reduce the potential bending moment between the tip and the side port. The cross sectional area of the side port is configured to be about equal to the cross sectional area of the lumen of the hollow needle.

U.S. Pat. No. 5,800,407 discloses an epidural catheter which combines the benefits of the open-end and closed-end three lateral holes type catheters. The epidural catheter is stated as being designed to include an end hole and a plurality of lateral holes. The combined end and multiple lateral holed epidural catheter of the present invention includes seven holes within a 1.5 cm head. The holes are positioned along the head such that one hole is provided at the tip, three lateral holes are circumferentially arranged about the head at approximately 1 mm from each other, and three additional holes are axially displaced from one another by approximately 4 mm. All the holes are within the 1.5 cm from the tip. The radius of each of the lateral holes is the same, thus, the anesthetic solution stream is the same from each hole.

A common procedure using a needle is a stellate ganglion sympathetic nerve block. This procedure is a chemical paralysis of the stellate ganglion by a local anesthetic agent injected in the vicinity of the ganglion. When a predetermined amount of blocking agent is correctly injected, the middle cervical ganglion, the intermediate ganglion, the stellate ganglion and the second, third, and fourth thoracic ganglia are anesthetized (“blocked”). Additionally, the superior cervical ganglion is also anesthetized because the nerve fibers that form this ganglion extend through the above-mentioned ganglia. Thus, when a stellate ganglion sympathetic block is correctly executed, the entire cervicothoracic portion of the sympathetic nervous system is blocked. Generally, for short time duration blocks, local anesthetic drugs such as novocaine, pontocaine, xylocaine, metycaime and intracaine, nupercaine and the like are used. For long time duration blocks, alcohol and phenol are generally used.

In various embodiments of procedures for blocks, a physician relies on superficial landmarks (i.e. bones, cartilage, muscles, tendons, and blood vessels which are near the skin surface) to locate the area of insertion of the needle. Deep landmarks (i.e. bones, periosteum, fascial planes, tendons, and blood vessels) that cannot be seen or palpated can only be felt with the tip or distal end of the needle. Thus, the accuracy in identifying these deep landmarks depends greatly on the educated touch of the physician

One of the safest ways to perform a stellate ganglion sympathetic block procedure is to place a patient in need thereof in a supine position, without a pillow, with the patient's head in a neutral/relaxed position. While standing on the side of the body that is to be blocked, the physician tactilely locates the cricoid cartilage. The needle is then inserted in a position approximately one finger breadth below the cricoid cartilage, between the carotid sheath and the trachea on the side to be blocked, while aiming slightly medially until the needle makes bony contact with the ventral lateral side of the body of the seventh cervical vertebra (the approximate location of the stellate ganglion). When the needle is in said position, the anesthesia is injected. However, since the inserted location of the needle is a matter of “feel” for the physician, it is common for the needle tip (distal end) to be placed such that the tip opening is under the anterior longitudinal ligament. This positioning may interfere with injection.

Therefore, it would be advantageous to design a needle, which can effectively inject anesthesia during the performance of a stellate ganglion sympathetic block whether or not the needle tip opening is under the anterior longitudinal ligament without a concern for an intraneural and/or an intravascular injection.

Various embodiments of the present invention comprise a sharp needle with an at least partially occluded, closed tip or distal portion and at least one side port or opening, and related methods. Various embodiments of the present invention provide for at least one benefit of enhanced injection characteristics, increased operational efficiency, reduced cost per unit, reduced incidence of injury through intraneural/intravascular injection, reduced incidence of injury through pricking/piercing, and/or the like.

Novel needles of various embodiments of the present invention are capable of being standard 20 or 22 gauge needles with a side port positioned at a predetermined distance from the occluded needle tip. Although it is understood that the side port can be placed at any distance from the occluded needle tip, various embodiments that would find wide applicability in various applications within the medical industry comprise distances of about 1 mm, about 1.5 mm, about 2 mm, and about 4 mm. These distances comprise primary sizes required in various applications such that the blocking agent is properly injected in a desired location through at least one side port. Further embodiments incorporate a weeping tip.

Further, various embodiments of the present invention further comprise methods of use. In an exemplary, non-limiting embodiment of a method of the present invention wherein at least a portion of a nervous system of a patient is to be blocked by a medicament, the method comprises the steps of placing the patient in a supine position or extended position; locating a site for injection; inserting at least a portion of the needle into the site; and injecting the medicament into the patient. Further embodiments comprise stimulating the site and/or ablating the site.

The invention surgical needle and/or surgical assemblage is ideally suited for injection into tissue of medicaments containing nucleic acid encoding a therapeutic agent (or cells containing such nucleic acid). For example, the invention needle (when attached to an appropriate catheter) or invention surgical assemblage can be used to inject medicament(s) into the wall of a beating heart or other internal organ, without substantial loss of the medicament at the surface of the body wall and without substantial damage to tissue at the injection site caused by injectate.

### BRIEF DESCRIPTION OF THE FIGURES

In order that the manner in which the above-recited and other enhancements and objects of the invention are obtained, a more particular description of the invention briefly described above will be rendered by reference to specific embodiments thereof which are illustrated in the appended drawings. Understanding that these drawings depict only typical embodiments of the invention and are therefore not to be considered limiting of its scope, the invention will be described with additional specificity and detail through the use of the accompanying drawings in which:

FIG. 1 (Prior Art) is an illustration of a distal portion of a prior art needle;

FIG. 2 is an illustration of a distal portion of a needle of an embodiment of the present invention;

FIG. 3 is an illustration of an alternate embodiment of a distal portion of a needle of an embodiment of the present invention;

FIG. 4 is an illustration of an alternate embodiment of a distal portion of a needle of an embodiment of the present invention;

FIG. 5 is an illustration of a cross section of embodiment of a surgical instrument of the present invention;

FIG. 6 is an illustration of an alternate embodiment of a surgical instrument of the present invention;

FIG. 7 is an illustration of an embodiment of a tip of the present invention as disclosed in FIG. 6 viewing the tip from axis A;

FIG. 8 is an alternate embodiment of a tip of the present invention;

FIG. 9 is an alternate embodiment of a tip of the present invention;

FIG. 10 is an alternate embodiment of a tip of the present invention;

FIG. 11 is a side perspective of the tip disclosed in FIG. 8;

FIG. 12 is a side perspective of the tip disclosed in FIG. 9; and,

FIG. 13 is a side perspective of the tip disclosed in FIG. 10.

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition.

As used herein the term, “air” means and refers to a gaseous mixture that comprises at least about 20 mole percent O₂.

As used herein, the term “attached,” or any conjugation thereof describes and refers to the at least partial connection of two items.

Exemplary, non-limiting embodiments of medical instrument, surgical instruments, and/or the like that can be modified according to various teachings of the present invention include, but are not limited to, U.S. Pat. No. 6,949,087; U.S. Pat. No. 6,855,132; U.S. Pat. No. 6,558,353; U.S. Pat. No. 6,547,769; U.S. Pat. No. 6,387,163; U.S. Pat. No. 6,245,044; U.S. Pat. No. 5,871,470; U.S. Pat. No. 5,865,806; U.S. Pat. No. 5,836,914; U.S. Pat. No. 5,817,074; U.S. Pat. No. 5,800,445; U.S. Pat. No. 5,730,749; U.S. Pat. No. 5,669,882; U.S. Pat. No. 5,628,734; U.S. Pat. No. 5,573,519; U.S. Pat. No. 5,571,091; U.S. Pat. No. 5,480,389; U.S. Pat. No. 5,466,225; U.S. Pat. No. 5,336,191; U.S. Pat. No. 5,312,360; U.S. Pat. No. 5,304,141; U.S. Pat. No. 5,250,035; U.S. Pat. No. 5,242,410; U.S. Pat. No. 5,106,376; U.S. Pat. No. 4,994,034; U.S. Pat. No. 4,973,313; U.S. Pat. No. 4,629,450; U.S. Pat. No. 4,317,445; U.S. Pat. No. 4,308,875; U.S. Pat. No. 4,230,123; U.S. Pat. No. 3,856,009; U.S. Pat. No. 3,565,074 and, U.S. Pat. No. 2,922,420, the contents of which are hereby incorporated by reference as if they were presented herein in their entirety. In general, any catheter may be used with the various embodiments of the present invention.

As used herein, a “fluid” is a continuous, amorphous substance whose molecules move freely past one another and that has the tendency to assume the shape of its container, for example, a liquid or a gas.

As used herein, the term “medicament(s)” means and refers to all types of substances (e.g., including solutions and suspensions) that have a beneficial, desired or therapeutic effect. Non-limiting examples of medicaments suitable for use in the invention methods include anesthesia, biologically active agents, such as small molecule drugs, proteinaceous substances, polynucleotides or nucleic acids (e.gJ heterologous DNA, or RNA) and vectors, liposomes, and the like, containing such nucleic acids or polynucleotides, as well as liquid preparations or formulations thereof.

As used herein, the term “medical instrument” means and refers to any item, instrument or structure capable of connecting to a catheter, such as, but not limited to a stimulation device, tubing, piping, a medicament delivery system, a meter, a liquid repository (such as an I.V. bag), a syringe, and/or the like.

As used herein, the term “stylet” means and refers to a small poniard.

Other than in the operating examples, or where otherwise indicated, all numbers expressing quantities of ingredients or reaction conditions used herein are to be understood as modified in all instances by the term “about”.

Various embodiments of the invention method and devices are designed for injection of minute amounts of fluid medicaments into tissue or a body wall, for example, an interior body wall. The therapeutic amount of the medicament to be administered according to the invention method will vary depending upon the therapeutic goal to be accomplished, the size and age of the subject, the pharmacokinetics of the injectate, and the like. However, a therapeutic amount according to the present invention is typically in the range from about 0.1 cc to about 5.0 cc.

Various other embodiments are designed for treatment of a target tissue(s). In an embodiment, treatment of a tissue comprises at least one of probing, ablation, stimulating, and/or the like. In general, treatments capable with various embodiments of the present invention can be any treatment common in the art and should not be limited by the present disclosure.

In general, various embodiments of a device of the present invention comprise surgical instruments and related methods with at least one benefit of enhanced injection characteristics, increased operational efficiency, reduced cost per unit, reduced incidence of injury through intraneural/intravascular injection, reduced incidence of injury through pricking/piercing, and/or the like.

More particularly, embodiments of the present invention generally comprise a surgical instrument comprising an elongated at least partially hollow shaft, generally extending from a proximal end to a distal end; a sharp needle at least partially occluded, closed distal end or tip end; a connector end; and, at least one side port located coaxially along the shaft. In further embodiments, the surgical instrument further comprises a medical instrument, i.e., any item, instrument or structure capable of connecting to a connector, such as, but not limited to a stimulation device, tubing, piping, a medicament delivery system, a meter, a liquid repository (such as an I.V. bag), a syringe, and/or the like connected to the connector end.

A tip end or distal end of the present invention generally comprises a shoulder along the juncture of the tip and the shaft; at least one generally beveled edge extending from the shoulder to the point of the tip. In an embodiment, a beveled edge extends circumferentially from about the shoulder to the point whereby the point of the tip is still capable of being inserted into at least one tissue of a patient, but the exposed edge or point is not as sharp as a needle point. In an alternate embodiment, the point of the tip is defined by a beveled edge extending from the shoulder to the point in a quadrangular arrangement, such that the beveled edges meet in a stealth arrangement whereby the point of the tip is still capable of being inserted into a tissue of a patient, but the exposed edge or point is not as sharp as a needle point. In an alternate embodiment, the point of the tip is defined by a beveled edge extending from the shoulder to the point in a triangular arrangement whereby the point of the tip is still capable of being inserted into a tissue of a patient, but the exposed edge or point is not as sharp as a needle point. In an alternate embodiment, the point of the tip is defined by a beveled edge extending from the shoulder to the point to form a blade whereby the point of the tip is still capable of being inserted into a tissue of a patient, but the exposed edge or point is not as sharp as a needle point. In an alternate embodiment, the point of the tip is defined by a beveled edge extending from the shoulder to the point whereby the point of the tip is still capable of being inserted into a tissue of a patient, but the exposed edge or point is not as sharp as a needle point. In general, a point associated with a tip of the present invention can be defined by any number of bevels or facets whereby the point of the tip is still capable of being inserted into a tissue of a patient, but the exposed edge or point is not as sharp as a needle point.

Angles of bevel for various embodiments can vary. In general, an angle of bevel can be from about 1° to about 90°. In an embodiment, by way of example, the angle of bevel is between about 15° and about 65°. In an alternate embodiment, again by way of example, the angle of bevel is about 45°. The angle of bevel is capable of being the same for all facets. However, in various embodiments, the angle of bevel varies for at least one facet as compared to the remaining facet(s). Design characteristics to consider when determining a proper bevel include, but are lot limited to, the tissue to be punctured, the delicate organs about the incision, the medicament to be administered, and/or the like.

Likewise, and related to the bevel angle is the length of a needle. For example, in an embodiment, an angle of 45° yields a tip length of about 0.05055 mm for a 20 gauge needle and approximately 0.03995 mm for the 22 gauge needle. However, tip lengths may vary according to the particular embodiment.

A side port of the present invention is generally a port extending from the exterior of the needle to the interior of the needle. The shape of a port can vary. In an embodiment, a port is circular. In an alternate embodiment, a port is ovular. In an alternate embodiment, a port is a quadrangular port, such as a rectangle or a square. In an alternate embodiment, the port is triangular. In general, ports of the present invention can be any shape sufficient to permit fluid aspiration therethrough.

A side port of the present invention may be further characterized by the associated edge of the port on the shaft. In an embodiment, a port has a slightly inwardly beveled edge extending from the exterior surface of the shaft to the interior surface. In an alternate embodiment, a port has a slightly outwardly beveled edge extending from the interior surface of the shaft to the exterior surface. In an embodiment, the degree of bevel can be used to change the pressure of the medicament as it enters the target tissue; facilitate a change in the degree of spread of the medicament; and, allow for a smooth surface as the shaft is inserted to the target tissue.

In various embodiments, a side port of the present invention can be located at any location about the needle. In an embodiment, the at least one side port is aligned with the point or edge of the distal portion of the needle. In an alternate embodiment the at least one side port is located 180-degrees from alignment with the point or edge of the distal portion of the needle.

Further embodiments of the present invention comprise a shaft with multiple ports arranged in any orientation about the shaft. Further embodiments comprise a porous portion, such that the port is one of multiple ports and a medicament introduced into the surgical instrument would tend to ooze out of the shaft into the target tissue. Accordingly, by way of example, an embodiment comprises an occluded needle with a weeping tip portion for microinjection of medicaments into a tissue. In an embodiment, again by way of example, the surgical instrument comprises a nonporous hollow needle shaft having a connector end adapted to mate with a surgical instrument, a porous distal portion in fluid-tight connection to the needle shaft, and a closed tip. The porous distal portion of the invention needle is adapted to cause a liquid injectate to weep or ooze therefrom multidirectionally under injection pressure while the distal portion and point of the needle are inserted into a tissue. In an embodiment, the porous distal portion oozes at a substantially uniform rate.

A cannula associated with various embodiments of a surgical instrument is generally a cylindrical structure extending from the connector to the tip end. In an embodiment, the shaft is of a generally constant circumference. The shaft is capable of being differentiated by an inside diameter and an outside diameter. In an embodiment, an outside diameter is between about 0.0355 to about 0.03600 mm and an inside diameter is between about 0.0230 to about 0.0245 mm. In an alternate embodiment, an outside diameter between about 0.0280 to 0.205 mm and an inside diameter between about 0.0155 to 0.0170 mm. Generally, an inside diameter and an outside diameter are capable of being any desired length.

Connector ends of the present invention comprise an attachment means for attaching the sharp needle at least partially occluded, closed distal end to the shaft. Further, connector ends of the present invention comprise an attachment means for attaching a medical instrument. The connection or connections at the connector end may be any type of connection common in the art, such as, for example, and not by way of limitation, a luer lock connector, a threaded attachment, an interference fit attachment, a clamp, a system utilizing a dowel, two or more of the aforesaid in combination, and/or the like.

Embodiments of connectors of the present invention optionally further comprise at least one wing. A wing of the present invention may be any structure. In an embodiment, the wing is an extension of the connector, such as, but not limited to, a rigid flap, a rigid bar, a flexible flap, a flexible bar, and/or the like. In further embodiments at least two wings extend from about the connector, such as, for example, and not by way of limitation, two wings extending evenly spaced circumferentially about the connector. Further embodiments have various numbers of wings extending from, attached to, or secured to the connector, in various arrangements.

In an embodiment, one wing extends from about the connector. A single wing connector or hub has certain benefits that are appealing for a variety of applications. It is common in the medical field that needles are inserted into patients for a variety of procedures. An issue that arises is how to secure or stabilize the needle before during and/or after insertion. A hemostat, pliers, and/or the like are often used to secure an area about the connector to secure it. However, the use of a hemostat, pliers, and/or the like is capable of occluding visualization down and/or about the needle and/or catheter. Further, the use of any metal tends to degrade the quality of images produced through NMR, X-ray, and/or the like. As well, the use of an instrument to secure the needle that is capable of interfering with an image through or about the needle is not desired in many applications. Accordingly, numerous procedures, such as, but not limited to, fluoroscopic guidance procedures, NMR procedures, X-ray procedures, direct viewing procedures, and/or the like would find a benefit in a single winged structure. Solutions in the art comprise attaching wings to the connector, in most cases, two wings. However, the inventors are unaware of the art field containing a surgical instrument comprising an elongated at least partially hollow shaft comprising a sharp needle at least partially occluded, closed distal end; a connector end; and, at least one side port located coaxially along the shaft, with a single wing. Double winged structures exist in the art, but are more expensive to manufacture, and the double wings do not add appreciable stability. Accordingly, benefits can be realized with a one winged design that can be secured by a hemostat, pliers and or the like, without requiring bending or manipulation of the needle and any associated connector and/or wing.

The inventor believes that the double winged structures were created so that, among other attributes, medical personnel could stabilize the needle by pinching the two wings together as the needle is inserted. However, pinching the two wings together creates inherent instability.

In various embodiments, a measurement that is capable of differentiating various embodiments of the present invention is the measurement from the shoulder of the tip to an edge of a port. In general, embodiments of the present invention comprise any shaft wherein the needle is not adjacent to a shoulder of the tip end. By not being adjacent means that a shoulder of a tip and a port on the shaft are at least separated by some distance. In an embodiment, by way of example, the distance is between about 0.01 mm to about 100.0 mm. In an alternate embodiment, again by way of example, the distance is between about 0.5 mm to about 75.0 mm. In an alternate embodiment, again by way of example, the distance is between about 1.0 mm to about 50.0 mm. In an alternate embodiment, again by way of example, the distance is between about 5.0 mm to about 25 mm. In general, the distance between the shoulder of the tip and the port of the associated shaft can vary as needed for the particular application.

Various embodiments of the present invention further comprise a wire or other means of conveying stimulation to a target tissue. In an embodiment, by way of example, the wire extends along the inside of the interior of the shaft from the connector to the tip. In an alternate embodiment, again by way of example, the wire is integral with the shaft. In an alternate embodiment, again by way of example, the wire extends along the outside of the shaft. It is preferred to have the wire secured to a surface so that the wire is not loose.

Design considerations that are capable of being implemented with various embodiments of the present invention include, but are not limited to designing the wire and connector such that they may be utilized as a plug and use type of arrangement. A plug and use arrangement is beneficial because it reduces the complexity of the device and reduces loose wires. In an embodiment, by way of example, the wire is formed into the connector such that when the connector is connected to another medical instrument, the wire is able to communicate with the instrument.

However, any connection common in the art that would allow the wire to communicate with a medical instrument is contemplated within the various embodiment of the present invention.

Further modifications of embodiments of a surgical instrument with a wire comprise the introduction of insulation about the cannula and/or wire. In general, any method of insulation or type of insulation could be used such as, but not limited to, a plastic, a metal, and/or the like.

Further modifications of embodiments of a surgical instrument with a wire comprise the introduction of a probe about the shaft and/or wire. Various probes capable of use with embodiments of the present invention include temperature probes, stimulation probes, cameras, and/or the like.

Accordingly, a further embodiment of the present invention comprises a surgical instrument comprising an elongated at least partially hollow shaft comprising a sharp, occluded needle means for insertion into a patient; a connector end connected through a connection to an instrument; and, at least one side port located coaxially along the shaft.

Also contemplated in various embodiments of the present invention are methods of use and manufacture of a surgical instrument of the present invention

An exemplary embodiment of a method of the present invention comprises a method for administering a medicament to a patient comprising the steps of locating a site for injection; inserting at least a portion of a surgical instrument into the site, the surgical instrument comprising an elongated at least partially hollow shaft having a sharp needle at least partially occluded, closed distal end; a connector end; and, at least one side port located coaxially along the shaft; and, injecting the medicament into the patient. In an embodiment, the medicament is anesthesia.

Further embodiments of a method of the present invention comprise stimulating a tissue. Yet further embodiments of a method of the present invention comprise ablating at least a portion of the tissue about a site. Other embodiments comprise probing a tissue. And yet further embodiments of a method of the present invention comprise preparing a patient for administering a medicament.

In an embodiment of an administration of a block, a method of the present invention comprises placing a patient in a supine position or extended position, without a pillow, with the patient's head in a neutral position. While standing on side of the body that is to be blocked, the physician tactilely locates the cricoid cartilage. The needle is then inserted in a position approximately one finger breadth below the cricoid cartilage, between the carotid sheath and the trachea on the side to be blocked, while aiming slightly medially until bony contact is made with the ventral lateral side of the body of the seventh cervical vertebra. When the needle is in said position, the anesthesia is injected. The new needle design allows for directional injection out the side port onto the surface of the anterior longitudinal ligament. This allows the anesthesia to spread onto the surface of the longus coli muscle toward the stellate ganglion. Thus, an injection via the side port will achieve an effective stellate ganglion sympathetic block.

In another embodiment according to the present invention, there are provided methods for injecting a medicament into tissue in a subject in need thereof. The invention injection method comprises inserting the distal portion of the invention needle into the tissue of the subject and causing a therapeutic amount of the medicament to ooze multidirectionally from the needle into the tissue without substantial leakage or loss of the medicament at the surface of the tissue. The invention method using the invention needle (or surgical assemblage) with porous distal portion is designed for injection of minute amounts of fluid into tissue or a body wall, hence the use of the term “microinjection” herein. However other amounts may also be injected.

In another embodiment according to the present invention, there are provided methods for injecting a medicament into a subject in need thereof, comprising inserting the distal portion of the invention needle into an interior body wall or tissue of the subject and applying sufficient pressure to a liquid medicament in fluid communication with the distal portion of the needle to expel the medicament such that the medicament weeps multidirectionally from the pores in the distal portion thereof into the interior body wall or tissue without substantial leakage or loss of the medicament at the surface of the body wall.

Various embodiments of the present methods are particularly useful for injecting medicament(s) into an interior body wall or tissue that is subject to motion, for example, the wall of a beating heart during electrophysiologic testing, transmyocardial revascularization, and the like.

In yet another embodiment, the present invention provides a method for injecting a medicament into tissue in a subject in need thereof comprising: inserting the distal portion of an invention needle into the tissue of the subject and causing a therapeutic amount of the medicament to ooze multidirectionally from the needle into the tissue without substantial damage to the tissue of the subject caused by injectate.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims which come within the meaning and range of equivalency of the claims are to be embraced within their scope. Further, all published documents, patents, and applications mentioned herein are hereby incorporated by reference, as if presented in their entirety herein.

### EXAMPLE

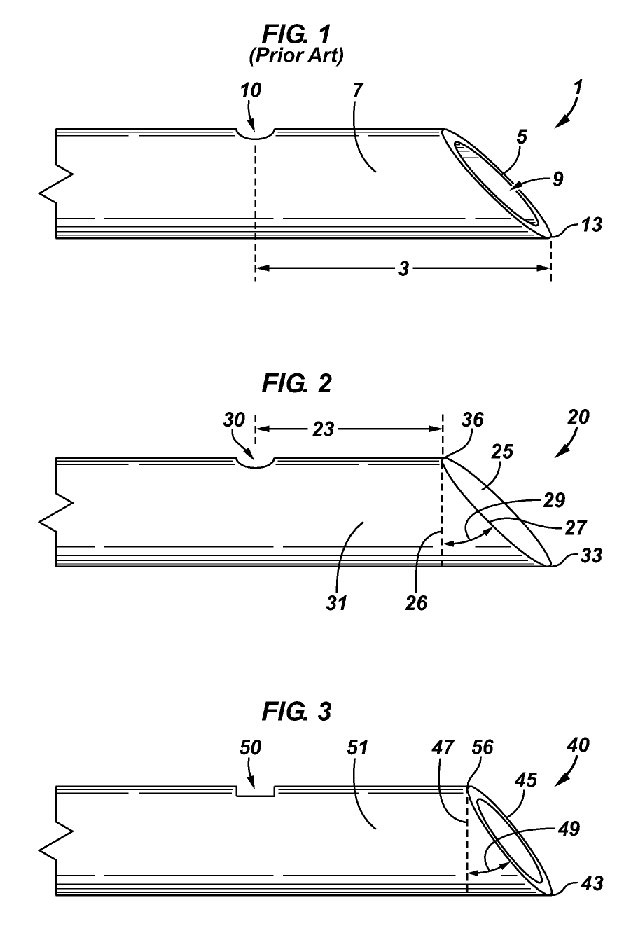

FIG. 1 (Prior Art) illustrates a prior art stellate ganglion sympathetic block surgical instrument 1 having a shaft 7, a needle tip 5, tip orifice 9 and a side port 10. Shaft 7 is further defined by a distance from the absolute tip of needle tip 5 to side port 10 and is represented by distance 3. In various embodiments, this prior art needle is generally between about 38 mm to 127 mm in length. The stellate ganglion sympathetic block surgical instrument 1 is described as, in various embodiments, a 20 or a 22 gauge needle. A 20 gauge needle has an outside diameter of about 0.0355 to 0.0360 mm and an inside diameter of about 0.0230 to 0.0245 mm. A 22 gauge needle has an outside diameter of about 0.0280 to 0.0205 mm and an inside diameter of about 0.0155 to 0.0170 mm. These prior art embodiments can be modified in accordance with the teachings of the present invention.

Now referring to FIG. 2, an illustration of a distal portion of a needle of an embodiment of the present invention, various enhancements of embodiments of the present invention are realized. Surgical instrument 20 comprises a sharp needle at least partially occluded, closed tip end 25, shaft 31, and a side port 30. Tip end 25 comprises a point 33 and a shoulder 27. The bevel of tip end 25 is represented by angle 29 measured by a line extending from tip edge 36 along shoulder 26. Needle is at least partially defined by measurement 23, the distance between point 33 and side port 30. Measurement 23 is 1.1 mm in this embodiment. A single bevel is illustrated terminating in tip 33.

Now referring to FIG. 3, an illustration of an alternate embodiment of a distal portion of a needle of an embodiment of the present invention, a more occluded embodiment of a needle with a different side port is illustrated. Surgical instrument 40 comprises a tip end 45, a shaft 51, and a side port 50. Side port 50 is a quadrangular structure in this embodiment. Angle 49 is smaller than angle 29 of FIG. 2, indicating that the bevel is shallower from tip edge 56 along shoulder 47. A single bevel is illustrated terminating in tip 43.

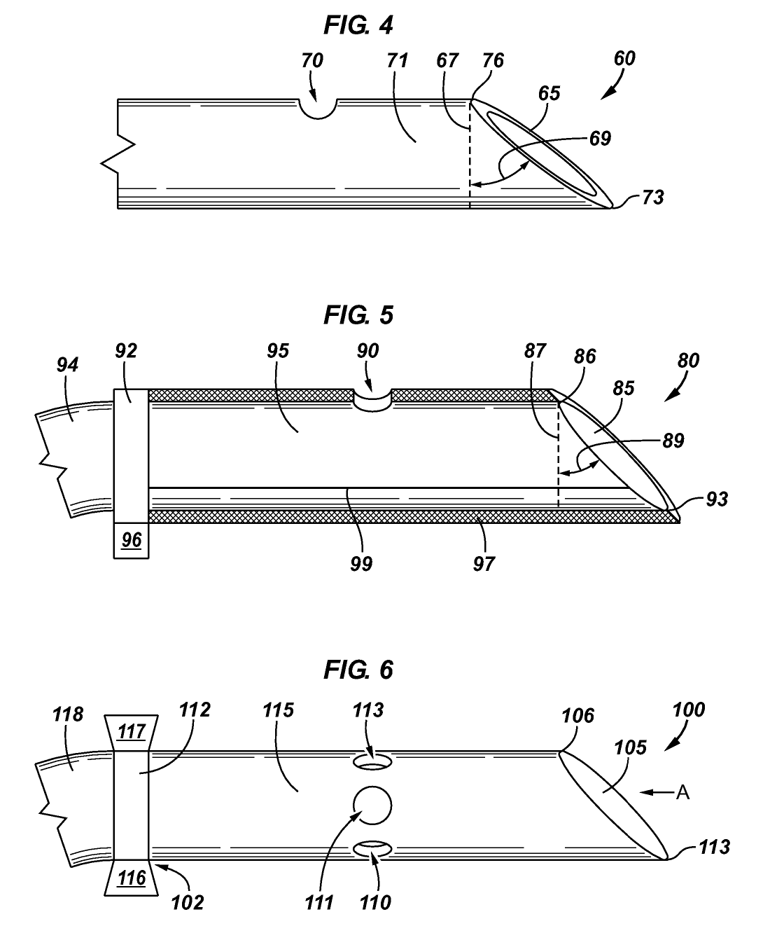

Now referring to FIG. 4, an illustration of an alternate embodiment of a distal portion of a needle of an embodiment of the present invention, a sharper embodiment of a thinner needle with a different side port is illustrated. Surgical instrument 60 comprises a tip end 65, a shaft 71, and a side port 70. Side port 70 is an ovular structure in this embodiment. Angle 69 is greater than angle 29 of FIG. 2, indicating that the bevel is steeper from tip edge 76 along shoulder 67. A single bevel is illustrated terminating in tip 73.

Now referring to FIG. 5, an illustration of a cross section of an embodiment of a surgical instrument of the present invention, an insulated embodiment is illustrated. Surgical instrument 80 comprises a tip end 85, shaft 95, side port 90, connector 92, wing 96, and medical instrument 94. Surgical instrument 80 further comprises a wire 99 extending along an interior surface of shaft 95 and an insulation 97 circumferentially surrounding shaft 95. Tip end 85 comprises tip edge 86, point 93, angle 89, and shoulder 87.

Now referring to FIG. 6, an illustration of an alternate embodiment of a surgical instrument of the present invention, an embodiment with multiple ports is illustrated. Surgical instrument 100 comprises a tip end 105, shaft 115, side port 110, side port 111, side port 113, connector 112, wing 116, wing 117, and medical instrument 118. Tip end 105 comprises tip edge 106 and point 113. In this embodiment, three ports and a double wing structure are illustrated.

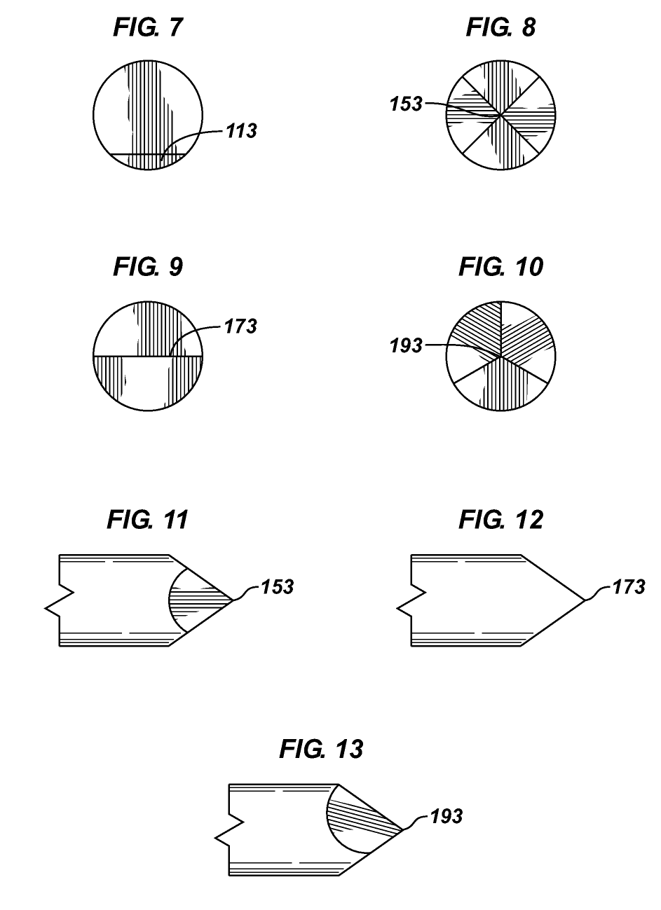

FIGS. 7, 8, 9, and 10 are illustrations of points of the present invention viewing along axis A of FIG. 6. FIG. 7 is an illustration of a view along axis A of FIG. 6 illustrating a single bevel terminating in point 113. FIG. 8 is an illustration of a quadrangular bevel terminating in point 153. FIG. 9 is an illustration of a wedge type bevel terminating at edge point 173. FIG. 10 is a triangular bevel terminating at point 193. FIGS. 11, 12, and 13 are side perspective view of FIGS. 8, 9, and 10.

What is claimed is:

1. A surgical instrument comprising:
an elongated at least partially hollow shaft comprising a sharp needle at least partially occluded, closed tip end;
a connector end; and,
at least one side port located coaxially along the shaft and not adjacent to a shoulder of said tip end.

2. The surgical instrument of clam 1, wherein the connector end further comprises at least one wing.

3. The surgical instrument of claim 1, further comprising at least a second side port located coaxially along the shaft and not adjacent to a shoulder of tip end.

4. The surgical instrument of claim 1, wherein the sharp needle at least partially occluded, closed distal end comprises at least two beveled edges.

5. The surgical instrument of claim 1, further comprising a wire secured to a surface of said needle.

6. The surgical instrument of claim 5, further comprising an insulation disposed about said surface of said needle.

7. The surgical instrument of claim 1, further comprising a probe disposed about a surface of said needle.

8. The surgical instrument of claim 5, wherein the wire is adapted to be connected about the connector with an instrument.

9. The surgical instrument of claim 8, wherein said connector is a plug and use connector.

10. The surgical instrument of claim 1, wherein the sharp needle at least partially occluded, closed tip end is at least partially rounded at the juncture of the tip end and the shaft.

11. A surgical instrument comprising:
an elongated at least partially hollow shaft comprising a sharp, occluded needle means for insertion into a patient wherein the sharp occluded needle means realizes at least one benefit of enhanced injection characteristics, increased operational efficiency, reduced cost per unit, reduced incidence of injury through intraneural/intravascular injection, reduced incidence of injury through pricking/piercing;
a connector end connected through a connection to an instrument; and,
at least one side port located coaxially along the shaft and not adjacent to a shoulder of said tip end.

12. The surgical instrument of claim 11, wherein the connection is a plug and use connection.

13. The surgical instrument of claim 11, further comprising at least one wing on said connector.

14. A method for administering a medicament to a patient comprising the steps of:
locating a site for injection;
inserting at least a portion of a surgical instrument into the site, the surgical instrument comprising an elongated at least partially occluded hollow shaft having a sharp needle at least partially occluded, closed distal end; a connector end; and, at least one side port located coaxially along the shaft and not adjacent to a shoulder of said tip end;
and, injecting the medicament into the patient.

15. The method of claim 14 further comprising the step of stimulating a tissue about the site.

16. The method of claim 15, wherein the step of stimulating said tissue comprises ablating at least a portion of said tissue.

17. The method of claim 14, wherein the medicament is anesthesia.

18. The method of claim 15, further comprising the step of preparing a patient for administering a medicament.
